(19) United States
(12) Patent Application Publication
(10) Pub. No.: US 2007/0077223 A1
(43) Pub. Date: Apr. 5, 2007

(54) Headwear with deodorizatnt function

(76) Inventor: Tung Hua Tai, Taipei (TW)

Correspondence Address: Tung Hua, Tai, P.O.Box 166-13, Taipei, 115, TW

(21) Appl. No.: 11/542,125

(22) Filed: Oct. 4, 2006

Related U.S. Application Data
(60) Provisional application No. 60/723,451, filed on Oct. 5, 2005.

(51) Int. Cl. A61L 9/00; A61K 36/899

(52) U.S. Cl. 424/76.2; 424/725.1

(57) ABSTRACT

A headwear with specific functions, including anti-bacteria, odor proofed, deodorant, warm-keeping, and so on, comprises a sweat band made by the fabric material, wherein at least one functional fiber is within a proper section of the sweat band, such as bamboo charcoal based fiber with deodorant anti-bacterial functions, or added with aromatic hydrocarbon to release aroma, and so on, thus, the sweat band can be as a functional sweat band, which can be used to keep fresh and clean within the headwear according to the functional fiber with anti-bacteria and deodorant.

### FIELD OF THE INVENTION

The present invention relates to a headwear, and more particularly to a headwear with sweat band including bamboo coal based activated carbon is, therefore, deodorant, odor proofed, warm-keeping, and so on. The present invention benefits from a provisional application date Oct. 5, 2005 assigned application No. as 60/723,451 in the USPTO.

### BACKGROUND OF THE INVENTION

Kyoto agreement intends to reduce the carbon dioxide emission around the world, Japanese expert transferred bamboo coal technique to Taiwan disclosed that when Thomas Edison invented electric lamp once used bamboo fiber originated from Kyoto instead of tungsten fuse, needless to say, it failed. But bamboo products mixed with nanometer technology may give a new life to the processed bamboo natural product.

Bamboo coal (or charcoal) based on activated coal now is gradually developed into a matured industrial technique in Taiwan in the shadow of annually imported ten million dollars anthracite product from China, such as Nin Xia coal mine. Since Taiwan still has 15,000 hectares bamboo forest, to reduce carbon dioxide emission including petroleum, anthracite etc., recently the competent authority in Taiwan found two kilogram of 4 year old Men-ChungChu species carbonized to produce methane totally can generate 5000 watt electricity, we can not estimate how many bamboo forest should be leveled instead of the valuable imported petroleum, or anthricate etc., but it is a new hope for useless bamboo forest turned into a valuable resource.

The same 4 year old Men-ChungChu species carbonized through 500 Celsius degrees treatment will be charcoal black, through 700-800 Celsius degrees treatment will be charcoal white, treated above Celsius 1000-1200 degrees, according to Taiwan Textile Research Institute expert opinion, it is said that up to 90% of the product can emit 4-1,000 micrometer (preferrable 4-14 micrometer) far infra red light. Thus, bamboo carbonized in a porous form through heating process, combining with nanometer technology through which the charcoal black or white ground to sub-nano size granules or added to the polyester fiber and then by drawwork to form estimated 2 denier 15 segments fiber. The finished fiber added with the component of bamboo charcoal in filament should be 1˜1.5%, but in staple 2˜3%. Surface area of an inch of said bamboo charcoal theoretically can be a football field size (2000 feet square), which is larger than the same provided by Japan (about 1200 feet square surface per inch). It is, therefore, with a humidity adjustable function, when the surrounding environment riches in rainfall or snow, the adsorption effect can be occurred to the bamboo charcoal, or when the surrounding environment is dry, the bamboo charcoal can release water molecules. By the same method, the deodorant effect can be expected from such a porous form bamboo charcoal. Even when it combined with a nanometer technology being ground granules or drawn fibers, the porous form still has the dampness proof and deodorant capacity. The same can even be used to retard the electromagnetic wave due to the dimension of the porous form. Bamboo charcoal usually selected from 4 years old Men ChungChu series processed product contains 80-90% carbon, the rest are volatile chemicals and ashes.

Bamboo charcoal based fiber can be used to produce a sweat band of a headwear of the present invention as desired. Since applications of headwear for modem people are popular, such as for sunshade, rain-fall protection, embellishing the apparel for people, however, the headwear maybe get dirtied by sweat or others after long time used, furthermore, that may brings bacteria and odor, which will be occurred annoyance for users.

And, often cleaning according to the headwear is troublesome for users also. During the cleaning, the headwear will be getting faded and deformed easily, such that the quality and lifetime of the headwear will surely be reduced. But, when a bamboo charcoal based activated carbon material is applied thereto, the headwear should be easily kept clean.

### SUMMARY OF THE INVENTION

Accordingly, how to design a headwear with deodorant function regard the previous mentioned shortcomings of the prior art, used for deodorant, odor proofed, anti-bacteria, warm-keeping, is the key point of the present invention.

It is a primary object of the present invention to provide a headwear, comprising a sweat band made by a fabric blended at least one functional fiber, i. e. bamboo charcoal based sub nano size fiber including anti-bacteria, odorant, odor proofed, and warm-keeping functions, such that the times of cleaning the headwear can be reduced, and the lifetime of that can be increased.

It is a secondary object of the present invention to provide a headwear, comprising a sweat band made by a fabric blended at least one functional fiber i.e. bamboo charcoal fiber to be used for clean and fresh within the headwear.

To achieve the previous mentioned objects, the present invention provides a headwear with deodorant function, comprising a sweat band therein, wherein the sweat band is made by a fabric, and the proper section of which comprises at least one functional fiber.

Since the sweat band is decorated within a headwear, it is hidden from view inside the headwear, but substantially decorated directly around the forehaead in contact with skin of the user, therefore, it should keep the user more comfortable no matter what kind of weather when putting on such a headwear.

### BRIEF DESCRIPTION OF THE DRAWINGS

It will be understood that the figures are not to scale since the individual layers are too thin and the thickness differences of various layers too great to permit depiction to scale.

FIG. 1 is an explosive view of a preferred embodiment of the present invention headwear with deodorant function;

FIG. 2 is a perspective view of a preferred embodiment of the present invention;

FIG. 3 is a perspective view of another preferred embodiment of the present invention;

FIG. 4 is a perspective view of another preferred embodiment of the present invention; and

FIG. 5 is a perspective view of another preferred embodiment of the present invention.

### DETAILED DESCRIPTION OF THE INVENTION

The structural features and the effects to be achieved may further be understood and appreciated by reference to the presently preferred embodiments together with the detailed description.

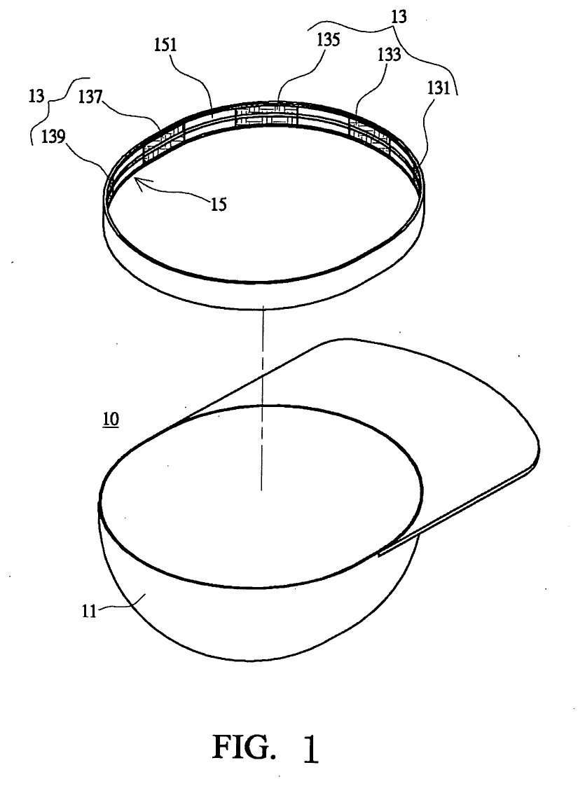

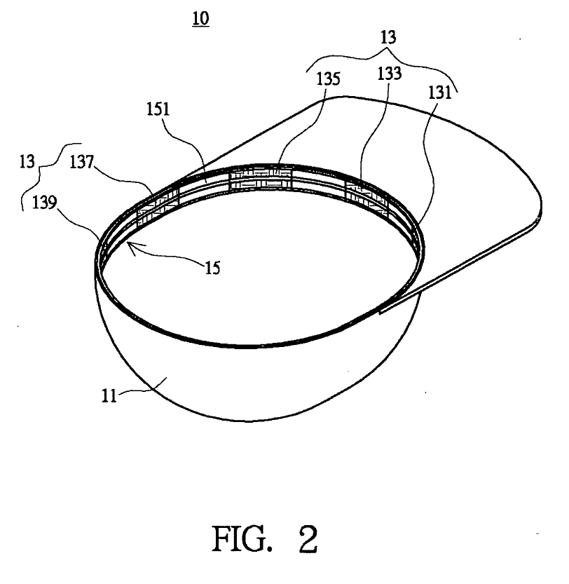

Firstly, referring to the FIG. 1 and FIG. 2, are respectively as an explosive view and a perspective view of a preferred embodiment of the present invention headwear with deodorant function. The headwear 10 comprises a crown 11 and a sweat band 15, wherein the sweat band 15 is made by a sweat band fabric 151, which can be blended at least one functional fiber i.e. resin based bamboo charcoal fiber 13, or at least one functional fiber 13 i.e. bamboo charcoal based fiber is formed thereon, by the way of weaving, knitting, and yarn blending, such that the sweat band 15 is resin based activated carbon with functions accordingly. Surely, the headwear 10 can be also with functions in accordance with the sweat band 15. And, the functional fiber 13 i.e. bamboo charcoal based fiber can be distributed interruptedly around the sweat band 15, such as the functional fibers (131, 133, 135, 137, 139) is distributed interruptedly around the proper area of the sweat band fabric, formed as annularity appearance, as shown in FIG. 1.

Wherein, the functional fiber 13 can be selected from a series of bamboo charcoal fiber, since the bamboo charcoal fiber the products passed through 500 or 700-800 or 1,000-1,200 Celsius degrees treatments, it may be attributed to charcoal black, charcoal white, up to 90% emitting 4˜1,000 micrometer far infrared light respectively. Therefore, it can be used as a colorful pigment to dye with selected color paint. A bamboo charcoal fiber made sweat band is hidden from view, stitched by silver ion fiber to an inner circle of a headwear, at least, between a crown and a visor. The silver ion fibers can be stitched to form at least four parallel lines along the inner circle of the crown, or the stitched silver ion fibers are arrayed intermittently around the inner circle spaced one another within one centimeter in parallel, due to the silver ion inhibits the growth of bacteria without destruction. The sweatband, at least, is decorated to the forehead of the users, directly in contact with skin of the user around forehead etc., when the surrounding environment is replete with moisture and odors, the bamboo charcoal may facilitate an adsorption with deodorant mechanism as a porous form fiber to reduce the headwear from moisture immersion or odor contamination, or the surrounding environment is dry, the water molecules released from the bamboo charcoal may facilitate the user kept comfortable and a suitable humidity. As known, the edible bamboo shoot must be through boiling water treatment due to it contains, at least, cyanide odor, therefore, an anti-bacterial fiber or an aromatic fiber, and a combination thereof for achieving the purpose of deodorant, odor proofed, warm-keeping, anti-bacteria, and so on can be achieved by such a sweat band. Since the bamboo charcoal based fiber mixed with nanometer technology can be shaped 2 denier 15 segments fiber, The finished fiber added with the component of bamboo charcoal in filament should be 1˜1.5%, but in staple 2˜3%, otherwise, the finished fiber will be coarse. By means of nano technology, bamboo charcoal being ground granules to sub-nano size added to the polyester fiber to form resin based bamboo charcoal fiber, or the bamboo charcoal mixed with, for example, 5˜20% component of silver ion to draw fiber. Other than drawwork, for example, the bamboo charcoal fiber 13 is formed by doping abrasive powder from bamboo charcoal during yarn forming process, that is, after reeling out, the function of bamboo charcoal can be with the yarn or a fabric.

The bamboo charcoal fiber is with functions of deodorant, odor proofed, moisture absorption, anti-bacteria, far infrared, electromagnetic isolation, warm-keeping, and so on, therefore, the yarn, fabric, sweat band, and headwear 10 having the bamboo charcoal included, are as the same functions surely.

Of course, once the functional fiber is selectively formed as an aromatic fiber, the yarn, fabric, sweat band, and headwear 10 having the aromatic fiber included, are with functions of deodorant, odor proofed, aromatic, and so on.

The composition of odorant fiber, odor proofed fiber, and anti-bacteria fiber can be selectively added with a metal ion fiber (as a silver ion fiber), chitin fiber, TiO2 fiber, and a combination thereof. According to improve the effect of moisture absorption for the sweat band 15, the functional fiber 13 can be selectively as a moisture absorption fiber also to achieve the purpose. And, the functional fiber 13 can be selectively added with a fiber doped a material of quaternary ammonium salt or nano-silver (organic/inorganic), and impregnating or spray coating into the functional fiber 13 to achieve the effect of anti-bacteria. Further, the cloth of sweat band 15 can be selected from such as a terry cloth, stretch yarn, or plain cloth.

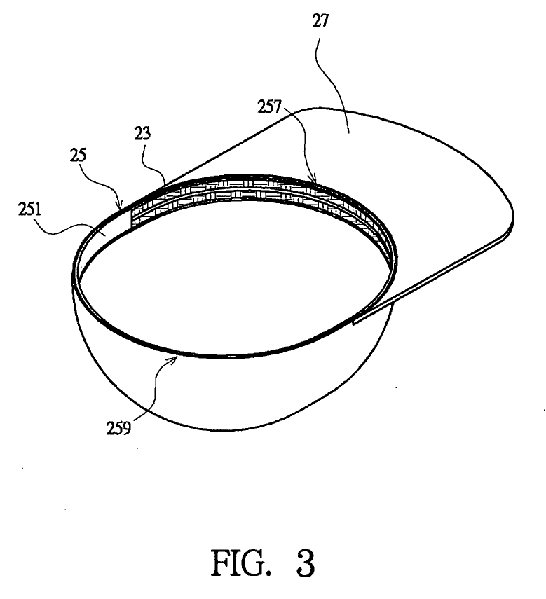

Referring to FIG. 3, is a perspective view of another preferred embodiment of the present invention. The sweat band 25 of the present invention is made by the sweat band fabric 251, wherein the left portion of the two sides of the sweat band fabric 251 is made by the functional fiber 23, such that can be formed as annularity appearance. For example, the functional fiber 23 can be provided, at least, the sweat band front edge 257 (or called as the first section of the sweat band) of the headwear visor 27 substantially among the visor and the crown (not shown). Besides, the sweat band rear edge 259, may be connected with the sweat band front edge 257 (or called as the second section of the sweat band), can be made by a general sweat band fabric 251, such that the usage efficiency of the functional fiber 23 can be improved, and further achieving the effect of deodorant, odor proofed, warm-keeping, anti-bacteria, and so on.

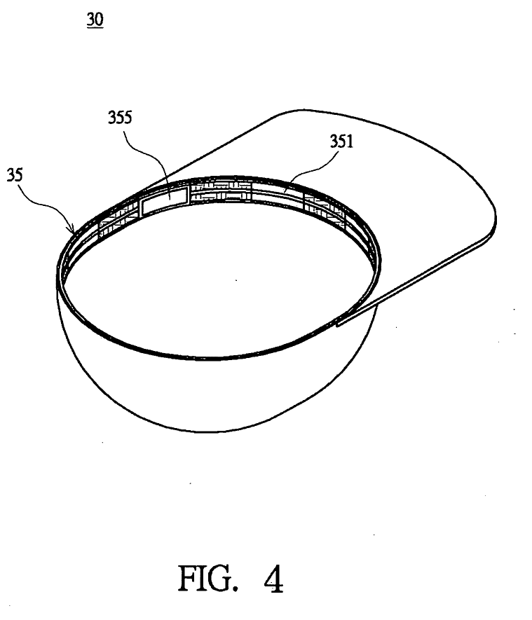

And, referring to FIG. 4, is a perspective view of another preferred embodiment of the present invention. The sweat band 35 further comprises a label 355 for conveniently providing the related information of the headwear 30 according to the manufacture, and easily understanding that for users. Due to an embodiment of the present invention, the label 355 can be provided on a proper section of the sweat band fabric 351. Usually, the label 355 is tagged to one end of the sweat band 35 around the forehead portion.

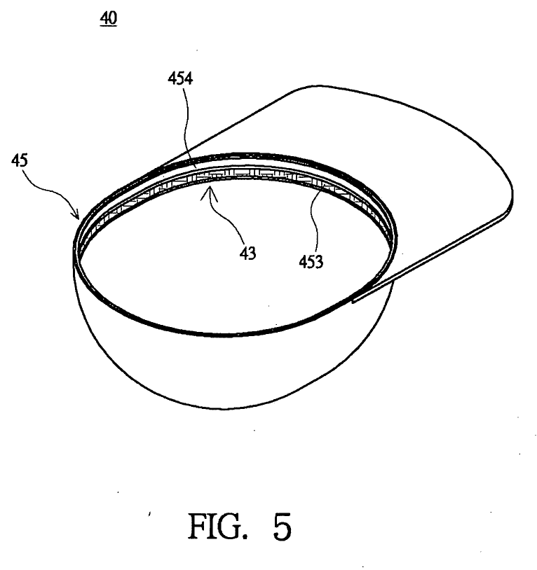

Finally, referring to FIG. 5, is a perspective view of another preferred embodiment of the present invention. The functional fiber 43 is distributed around the proper section of the sweat band 45, and formed as an annularity appearance. For example, the functional fiber 43 is provided on the upper annularity area 453 of the sweat band 45, once a user wears the headwear 40, the functional fiber 43 can be used to take care of the sweat from the head while the user is perspiring, such that the perspiring from users can be taken care and the material amount of the functional fiber 43 can be helpful retrenched. Of course, the functional fiber 43 can be also provided on the lower annularity area 454 of the sweat band 45, or formed as annularity appearance on the proper section of the sweat band 45.

The foregoing description is merely one embodiment of present invention and not considered as restrictive. All equivalent variations and modifications in process, method, feature, and spirit in accordance with the appended claims may be made without in any way from the scope of the invention.

I claim:

1. A headwear with deodorant function, comprising a sweat band therein, wherein said sweat band is made by a fabric, hidden from view stitched inside a headwear, at least, between a crown and a visor directly surround a forehead portion of a user, proper section of which comprises at least one functional fiber i.e. resin based bamboo charcoal fiber through drawwork, a finished fiber added with the component of bamboo charcoal in filament is 11.5%, but in staple 2˜3%.

2. The headwear of claim 1, wherein said functional fiber can be added with a bamboo charcoal fiber selectively added with a deodorant fiber, an anti-bacterial fiber, an aromatic fiber, a metal ion fiber, a chitin fiber, a moisture absorption fiber, a TiO2 fiber, and a combination thereof.

3. The headwear of claim 2, wherein said metal ion fiber is a silver ion fiber.

4. The headwear of claim 1, wherein said sweat band further comprises a label provided on a proper section of said sweat band.

5. The headwear of claim 1, wherein said bamboo charcoal under 500 Celsius degrees treatment so as to obtain a charcoal black.

6. The headwear of claim 1, wherein said bamboo charcoal under 700-800 Celsius degrees treatment to obtain charcoal white.

7. The headwear of claim 1, wherein said bamboo charcoal under 1000-1200 Celsius degrees treatment, up to 90% emit far infra red light wave from 4 to 1,000 micrometer.

8. The headwear of claim 1, wherein said resin based bamboo charcoal fiber is 2 denier, 15 segments.

9. The headwear of claim 1, wherein said cloth of sweat band selected from terry cloth, stretch yarn, plain cloth.
